(19) United States
(12) Patent Application Publication
(10) Pub. No.: US 2025/0276304 A1
(43) Pub. Date: Sep. 4, 2025

(54) CATALYST COMPOSITIONS AND METHODS OF PREPARATION AND USE THEREOF

(71) Applicants: BASF Corporation, Florham Park, NJ (US); THE REGENTS OF THE UNIVERSITY OF CALIFORNIA, Oakland, CA (US)

(72) Inventors: Joseph C. Dellamorte, Beachwood, OH (US); Enrique Iglesia, Berkeley, CA (US); Teng Fu, Shanghai (CN); Biswanath Dutta, Berkeley, CA (US)

(21) Appl. No.: 19/211,400

(22) Filed: May 19, 2025

Related U.S. Application Data
(62) Division of application No. 17/800,803, filed on Aug. 18, 2022, now Pat. No. 12,330,140.
(60) Provisional application No. 62/981,655, filed on Feb. 26, 2020.

(51) Int. Cl. B01J 21/06; B01J 23/10; B01J 35/50; B01J 35/51; B01J 35/61; B01J 37/18; C01G 25/02; C07C 5/333

(52) U.S. Cl. CPC B01J 21/066; B01J 23/10; B01J 35/50; B01J 35/51; B01J 35/612; B01J 35/613; B01J 35/615; B01J 37/18; C01G 25/02; C07C 5/3332; C01P 2002/76; C01P 2006/12; C07C 2521/06

(57) ABSTRACT

Disclosed are methods of dehydrogenating a light alkane gas (and/or light alkene gas), which include adding hydrogen (H₂) to the light alkane gas (and/or light alkene gas) in the presence of a catalyst composition containing zirconium oxide. Also disclosed are catalyst compositions containing zirconium oxide and methods of preparation thereof, where the catalyst compositions are useful in methods of dehydrogenating light alkane gas.

### CROSS-REFERENCE TO RELATED APPLICATIONS

The present application is a continuation of U.S. Non-Provisional patent application Ser. No. 17/800,803, filed on Aug. 18, 2022, which is a national stage entry under 35 U.S.C. § 371 of International Patent Application No. PCT/US2021/017072, filed on Feb. 8, 2021, which claims the benefit of priority of U.S. Provisional Patent Application No. 62/981,655, filed on Feb. 26, 2020, the disclosures of which are hereby incorporated by reference herein in their entireties.

### FIELD

This disclosure relates to methods of using catalyst compositions for dehydrogenation of light alkane gas (and/or light alkene gas). The disclosure also relates to catalyst compositions, for example, that can be used in such dehydrogenation reactions and methods of preparation thereof.

### BACKGROUND

Catalytic dehydrogenation of alkanes is an efficient conversion technology for the production of alkenes as compared to traditional petroleum-based processes such as fluid catalytic cracking and thermal steam cracking. Traditional processes suffer from high-energy demand, dwindling petroleum reserve and low selectivity toward any particular target alkene. Catalytic dehydrogenation is also more environmentally friendly than oil-based processes because it uses natural gas and shale gas as feedstock, which contain fewer impurities.

Two industrial processes for the production of light alkene using dehydrogenation are 1) the chromia-alumina-based Catofin® process; and 2) the Pt—Sn-based Oleflex™ process. However, the use of chromium (Cr) and platinum (Pt) based catalysts presents environmental and health issues. For example, according to the Occupational Safety and Health Administration (OSHA), human exposure to chromium (VI) (Cr⁶⁺) may cause serious health issues such as lung cancer.

Platinum-based catalysts used for dehydrogenation of alkanes can include Pt deposited alone, or in combination with another material such as tin (Sn), on an inactive support. The inactive support can be, for example, a ZrO₂ support that is inactive under the reaction conditions that cause the Pt or Pt—Sn to be active. However, the re-dispersion of Pt in such catalysts often requires the addition of chlorine-based compounds during the catalyst regeneration process, which is ecologically harmful. Moreover, such Pt-based catalysts are sensitive to trace impurities and very costly.

There is a need for methods and catalyst compositions that inhibit deactivation of the catalyst composition and at same time maintain a considerable dehydrogenation activity. There is a further need for catalyst compositions that are free of Cr and precious metals, such as Pt, and that increase the safety and sustainability of the dehydrogenation process. The use of such catalyst compositions in the dehydrogenation of light alkanes (and/or alkenes) could improve the total yield of dehydrogenation products within one cycle and require less frequent catalyst regenerations.

### BRIEF DESCRIPTION OF THE DRAWINGS

FIG. 1 shows an X-ray diffraction (XRD) spectrum of a catalyst composition comprising zirconium oxide (ZrO₂) according to embodiments.

FIG. 2a shows the dehydrogenation rate per mass of catalyst (r_{d}) and the selectivity of the dehydrogenation reaction (ratio between dehydrogenation and cracking rate, r_{d}/r_{c}) during a propane dehydrogenation (PDH) reaction on a pretreated ZrO₂ catalyst.

FIG. 2b shows the dehydrogenation rate per mass of catalyst (r_{d}) and selectivity of the dehydrogenation reaction (ratio between dehydrogenation and cracking rate, r_{d}/r_{c}) during a PDH reaction on a pretreated ZrO₂ catalyst.

FIG. 3 shows the dehydrogenation rate per mass of catalyst (r_{d}) during a PDH reaction on a pretreated ZrO₂ catalyst.

FIG. 4 shows the dehydrogenation rate per mass of catalyst with additional H₂ present (r_{d}) during a PDH reaction on a ZrO₂ catalyst with O₂/He pretreatment at different temperatures.

FIG. 5 shows the CO₂ generation rate during a re-oxidation process after a PDH reaction with and without the presence of additional H₂ at 873 K on ZrO2 catalyst.

FIG. 6 shows the dehydrogenation rate per mass of catalyst (r_{d}) during a PDH reaction.

FIG. 7(a) shows the dehydrogenation rate per mass of catalyst (r_{d}) during a PDH reaction on a ZrO₂ catalyst as a function of C₃H₈ partial pressure.

FIG. 7(b) shows the dehydrogenation rate per mass of catalyst (r_{d}) during a PDH reaction.

FIG. 8 shows a comparison of initial dehydrogenation rate of propane among ZrO₂ catalysts.

### BRIEF SUMMARY

According to various embodiments, disclosed herein are catalyst compositions, comprising zirconium oxide (ZrO₂), wherein the catalyst compositions are free of at least one of chromium and a precious metal (e.g., Pt, gold, silver, copper, palladium, etc.). In embodiments, the catalyst compositions are free of chromium and platinum.

Further disclosed herein, according to various embodiments, is a method of preparing a catalyst composition, comprising combining ZrO(NO₃)₂xH₂O, water and urea at a temperature of about 20° C. to about 25° C. to form a ZrO(NO₃)₂ solution; and crystallizing the ZrO(NO₃)₂ solution at an elevated temperature under an autogenous pressure for about 2 h to about 36 h to form the catalyst composition.

According to further embodiments, disclosed herein is a method for dehydrogenating a light alkane gas (and/or a light alkene gas), comprising: combining hydrogen (H₂) with the light alkane gas (and/or light alkene gas) in the presence of a catalyst composition comprising zirconium oxide (ZrO₂).

### DETAILED DESCRIPTION

Described herein are methods of using catalyst compositions for the dehydrogenation of light alkane gases (and/or light alkene gases). Also disclosed are catalyst compositions for such dehydrogenation reactions and methods of preparation thereof. It is to be understood that the invention is not limited to the details of construction or process steps set forth in the following description. The invention is capable of other embodiments and of being practiced or being carried out in a variety of ways.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

As used herein, the singular forms “a,” “an,” and “the” include plural references unless the context clearly indicates otherwise. Thus, for example, reference to “a catalyst material” includes a single catalyst material as well as a mixture of two or more different catalyst materials.

As used herein, the term “about” in connection with a measured quantity, refers to the normal variations in that measured quantity as expected by one of ordinary skill in the art in making the measurement and exercising a level of care commensurate with the objective of measurement and the precision of the measuring equipment. In certain embodiments, the term “about” includes the recited number±10%, such that “about 10” would include from 9 to 11.

The term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that. In certain embodiments, the term “at least about” includes the recited number minus 10% and any quantity that is higher such that “at least about 10” would include 9 and anything greater than 9. This term can also be expressed as “about 10 or more.” Similarly, the term “less than about” typically includes the recited number plus 10% and any quantity that is lower such that “less than about 10” would include 11 and anything less than 11. This term can also be expressed as “about 10 or less.”

Unless otherwise indicated, all parts and percentages are by weight except that all parts and percentages of gas are by volume. Weight percent (wt %), if not otherwise indicated, is based on an entire composition free of any volatiles, that is, based on dry solids content. Volume percent (vol %), if not otherwise indicated, is based on the total volume of the gas.

Although the disclosure herein is with reference to particular embodiments, it is to be understood that these embodiments are merely illustrative of the principles and applications of the invention. It will be apparent to those skilled in the art that various modifications and variations can be made to the compositions and methods without departing from the spirit and scope of the invention. Thus, it is intended that the invention include modifications and variations that are within the scope of the appended claims and their equivalents.

Disclosed herein are methods of using a catalyst composition comprising zirconium oxide for dehydrogenation of light alkane gas (and/or light alkene gas) in the presence of hydrogen gas (H₂). Also disclosed are catalyst compositions, for example, that can be used in the dehydrogenation reaction, and methods of preparing such compositions. As a non-toxic, earth-abundant and low-cost oxide material, ZrO₂ (e.g., having a small crystallite size of less than about 10 nm, or less than about 20 nm, or less than about 30 nm, or less than about 50 nm, or less than about 1 μm) exhibits a high dehydrogenation rate (r_{d}) during propane dehydrogenation reactions. Pre-treating a standard ZrO₂ catalyst (e.g., precipitated using methods known to those of ordinary skill in the art) with carbon monoxide (CO) (e.g., for 30 min at 823 K) prior to use in a dehydrogenation reaction can result in a propane dehydrogenation rate of about 84 mol kg⁻¹ h⁻¹, which is comparable to dehydrogenation rates exhibited with Pt-based materials. Without being bound to any particular theory, it may be that coordinatively unsaturated Zr sites (Zr_{cus}) are the active structures in ZrO₂ catalysts. With only a light alkane gas, such as propane (C₃H₈), in the inlet stream to a dehydrogenation process, the ZrO₂ catalyst may exhibit a half-life of only, for example, 0.2 h at 873 K, and rapid deactivation attributable to the formation of carbonaceous species on the surface of the ZrO₂. Regeneration of the ZrO₂ catalyst may require frequent cyclic operations based on dehydrogenation reactions and subsequent re-oxidation processes (e.g., in the operation of cycle Catofin-type processes). That is, while ZrO₂ catalysts exhibit favorable dehydrogenation activity during dehydrogenation reactions, they can suffer from rapid deactivation due to coke formation.

During investigations of light alkane gas dehydrogenation activity over a catalyst composition comprising ZrO₂ according to embodiments, it has been found that by co-feeding H₂ with the light alkane gas during the dehydrogenation reaction, the stability of the catalyst can be significantly increased. As set forth in the examples below, further studies have been conducted to verify this observation and to demonstrate the specific effect of H₂ on the activity of the catalyst composition comprising ZrO₂ during a light alkane dehydrogenation reaction while the bulk crystallite structure of the catalyst composition has been preserved during the reaction. According to various embodiments, the purposeful addition of H₂ to light alkane reactants during dehydrogenation reactions on catalyst compositions comprising ZrO₂ as the active material, leads to significant stability improvements, with half-life improvements of 100-fold upon addition of the H₂. Adding or co-feeding the H₂ with the light alkane gas can include introducing the H₂ at a pressure of about 1 kPa to about 100 kPa, or at a molar ratio of H₂ to light alkane gas of about 1:100 to about 1:1. In embodiments, the H₂ and/or reactor can be at a reaction temperature of about 500 K to about 1000 K, or about 873 K. The increase in stability may be attributed to the influence of H₂ on the formation of carbonaceous species.

To stabilize the catalyst composition comprising ZrO₂, hydrogen (H₂) (e.g., about 2-20 kPa) can be co-fed with the light alkane gas (and/or light alkene gas) during the dehydrogenation reaction. A significant increase in the stability of the catalyst composition comprising ZrO₂ can be achieved with more than a 100-fold increase in the catalyst's half-life. Without being bound by any particular theory, this increase in stability may be attributable to two factors: 1) the continuous regeneration of Zr active sites; and 2) the inhibition of coke formation via hydrogenative removal. As discussed above, the activity of catalyst compositions comprising ZrO₂ may be attributable to Zr surface-active sites on which light alkane molecules can be activated and dehydrogenated to alkenes or which could lead to the formation or organic species that themselves can act as a reaction-derived catalytic function on zirconia surfaces. A higher initial dehydrogenation rate also can be achieved through a pretreatment with a reducing gas (e.g., H₂, CO, NH₃, etc.) at a corresponding reaction temperature, possibly suggesting the generation of Zr surface-active sites as a result of the reductive pre-treatment. By co-feeding H₂ with the light alkane gas (and/or light alkene gas) during the dehydrogenation reaction, it may be that the Zr surface-active sites are continuously regenerated so that loss of active sites due to coke formation are compensated for accordingly.

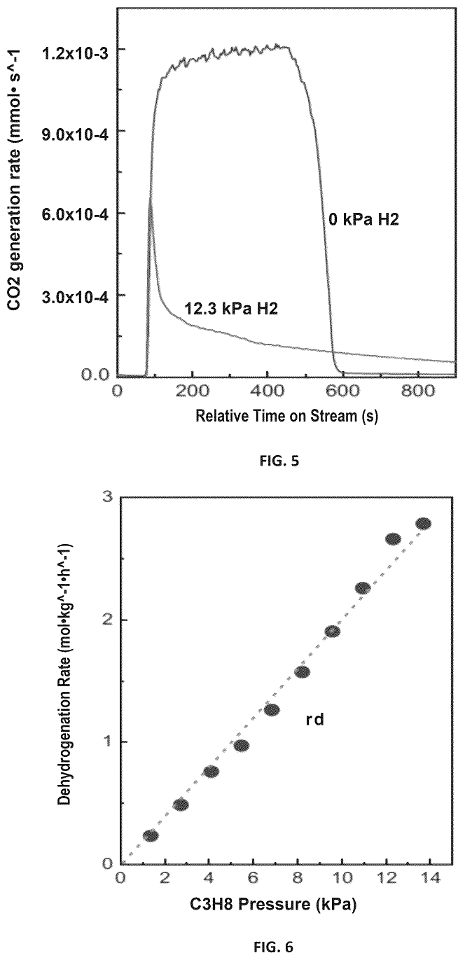

An inhibition effect on coke formation may be achieved by co-feeding H₂ with the light alkane gas (and/or light alkene gas) during dehydrogenation. Quantification of carbon dioxide (CO₂) generated during a reoxidation process demonstrates an 80% decrease in total coke amount during a propane dehydrogenation reaction in the presence of H₂ as will be discussed in more detail below with respect to FIG. 5. Co-feeding H₂ with the light alkane gas (and/or light alkene gas) can maintain and/or increase the number of active sites in a catalyst composition comprising ZrO₂ and while simultaneously inhibiting coke formation, resulting in improved stability of catalyst compositions for light alkane gas (and/or light alkene gas) dehydrogenation reactions.

Without being bound by any particular theory, the continuous generation of Zr surface-active sites (e.g., Zr_{cus}) may be achieved as the H₂ removes lattice oxygen from the surface of the catalyst composition comprising ZrO₂. As discussed above, pretreating the catalyst compositions using a reducing gas (e.g., H₂, CO, NH₃, etc.) is believed to generate Zr surface-active sites and can result in a higher initial dehydrogenation rate (r_{d}). Nonetheless, it has been observed that a continuous decrease in dehydrogenation rate may still exist on reduced catalyst compositions comprising ZrO₂ during the dehydrogenation reaction without the presence of additional H₂ due to the formation of carbonaceous species. The formation of carbonaceous species on the catalyst surface under reaction conditions has been identified as a main cause of deactivation for most all dehydrogenation catalysts. These carbonaceous species can include hydrogen-poor organic residues that block active sites by hindering contact with reactants and removal of products. Without being bound by any particular theory, it may be that the addition of H₂ during the dehydrogenation reaction generates Zr active sites (e.g., Zr_{cus} active sites) by removing surface lattice oxygen, such that the lost active sites covered by carbonaceous species may be compensated during the dehydrogenation reaction; when all of the surface lattice oxygen is removed, this mechanism may change. In embodiments, the H₂ can be present in an amount sufficient to remove surface lattice oxygen from the catalyst composition and to generate and/or reactivate surface-active sites. For example, the H₂ can be present in an amount of H₂ per mass of catalyst of about 1 mol kg⁻¹ h⁻¹ to about 100 mol kg⁻¹ h⁻¹, or about 55 mol kg⁻¹ h⁻¹.

When comparing the amount of carbon dioxide (CO₂) generated during the re-oxidation process, a decrease (e.g., 80%) in the amount of carbonaceous species can be observed when H₂ (e.g., 12.3 kPa) is added to the light alkane inlet stream at, for example, a temperature of about 873 K. The decrease in the amount of carbonaceous species formed can be attributable to the inhibited adsorption of certain polymerization intermediates (e.g., C₂H₄, C₃H₆, etc.) as the catalyst surface is populated and present in equilibrium with the added H₂.

### Catalyst Compositions

Catalyst compositions as described herein are useful in dehydrogenation reactions, for example, to dehydrogenate light alkane gases to form alkenes. In embodiments, the catalyst compositions can also be used to dehydrogenate light alkene gases to form alkadienes. According to embodiments, the catalyst compositions can comprise ZrO₂. The ZrO₂ is the active catalyst material in the dehydrogenation reactions according to various embodiments disclosed herein. In embodiments, the ZrO₂ is not inactive or an inactive support; rather it is the active catalyst material.

As will be discussed in more detail below, the catalyst composition may comprise an additive, such as yttrium, to increase the sintering stability and thus preserve the tetragonal phase of ZrO₂. The catalyst composition may also be free of at least one of chromium and a precious metal. Non-limiting examples of precious metals include platinum (Pt), gold (Au), silver (Ag), copper (Cu), palladium (Pd) and combinations thereof. In embodiments, the catalyst composition is free of both chromium and platinum. Catalyst compositions as described herein may present fewer risks to human health and the environment than other known catalysts, e.g., chromium- and platinum-based catalysts, used for dehydrogenation.

In embodiments, the catalyst composition can include at least about 50 wt % ZrO₂, at least about 60 wt % ZrO₂, at least about 70 wt % ZrO₂, at least about 80 wt % ZrO₂, at least about 90 wt % ZrO₂, or about 85 wt % ZrO₂, about 90 wt % ZrO₂, about 95 wt % ZrO₂, about 96 wt % ZrO₂, about 97 wt % ZrO₂, about 98 wt % ZrO₂, about 99 wt % ZrO₂, about 99.5 wt % ZrO₂, or about 100 wt % ZrO₂. In embodiments, the catalyst composition can include about 50 wt % to about 100 wt % ZrO₂, or about 60 wt % to about 100 wt % ZrO₂, or about 70 wt % to about 100 wt % ZrO₂, about 80 wt % to about 100 wt % ZrO₂, or about 90 wt % to about 100 wt % ZrO₂.

Catalyst compositions according to embodiments herein can contain a plurality of surface-active sites. Without being bound by any particular theory, for catalyst compositions comprising ZrO₂, it may be that the surface-active sites are coordinatively unsaturated Zr sites (Zr_{cus}). According to embodiments, catalyst compositions comprising ZrO₂ comprise a plurality of Zr_{cus} surface-active sites.

The catalyst compositions can be measured by X-ray diffraction (XRD), e.g., using a Siemens D5000 unit at ambient temperature with Cu Kα radiation and a scan rate of 0.033° s⁻¹ to determine the crystalline structure of the catalyst composition. Catalyst compositions comprising ZrO₂ in embodiments disclosed herein can be in at least one of the monoclinic phase and the tetragonal phase, for example, as measured by XRD as described in Zhang, et al., The Effect of Phase Composition and Crystallite Size on Activity and Selectivity of ZrO₂ in Non-Oxidative Propane Dehydrogenation, J. of Catalysis, 371, 313-324 (2019), which is incorporated herein by reference in its entirety. In embodiments, the catalyst composition comprises ZrO₂ in the monoclinic phase.

The Brunauer, Emmett and Teller (BET) surface area of the catalyst composition can be measured using nitrogen (N₂) physisorption uptake at its normal boiling point in a surface analyzer (e.g., a Quantasorb® 6 Surface Analyzer by Quantachrome® Corp.). The BET surface area can be measured as set forth in Otroshchenko, et al., Zro₂-Based Unconventional Catalysts for Non-Oxidative Propane Dehydrogenation: Factors Determining Catalytic Activity, J. of Catalysis, 348, 282-290 (2017), which is incorporated herein by reference in its entirety. Another suitable method of measuring the BET surface area is set forth in ASTM D3663-03 (2008), which is incorporated by reference herein in its entirety. Catalyst compositions comprising ZrO₂ as described herein can have a BET surface area of about 1 m² g⁻¹ to about 100 m² g⁻¹, or about 10 m² g⁻¹ to about 90 m² g⁻¹, or about 20 m² g⁻¹ to about 80 m² g⁻¹, 30 m² g⁻¹ to about 70 m² g⁻¹, 40 m² g⁻¹ to about 60 m² g⁻¹, or about 40 m² g⁻¹, or about 45 m² g⁻¹, or about 50 m² g⁻¹ as measured by a surface analyzer as described above.

According to various embodiments, the catalyst composition comprising ZrO₂ can further include a rare earth metal. In embodiments, the rare earth metal can include at least one lanthanide metal, an oxide thereof and combinations thereof. According to embodiments, the rare earth metal can be at least one of yttrium (Y), erbium (Er), cerium (Ce), dysprosium (Dy), gadolinium (Gd), lanthanum (La), neodymium (Nd), samarium (Sm), ytterbium (Yb), oxides thereof and mixtures thereof. In embodiments, the catalyst composition can include about 0.5 wt % to about 50 wt %, or about 1 wt % to about 40 wt %, or about 2 wt % to about 30 wt %, or about 3 wt % to about 25 wt %, or about 4 wt % to about 20 wt %, or about 5 wt % to about 15 wt %, or about 1 wt % to about 12 wt %, or about 2 wt % to about 10 wt % of the rare earth metal, an oxide thereof or mixtures thereof. In certain embodiments, the catalyst composition can include ZrO₂ stabilized with an yttrium dopant. The yttrium can increase the surface area of ZrO₂ and stabilizes the pure tetragonal phase of the ZrO₂. Such catalyst compositions can comprise about 85 wt % to about 99.5 wt % ZrO₂ and about 0.5 wt % to about 15 wt % Y₂O₃ and/or an atomic ratio of Y/Zr of greater than 0 to about 0.2. The catalyst composition comprising the yttria-stabilized zirconia (YSZ) can have a BET surface area of about 40 m² g⁻¹ to about 110 m² g⁻¹.

According to various embodiments, a catalyst composition comprising ZrO₂ can be treated with a pretreatment gas. Pretreating the catalyst composition can increase the number of surface-active sites on the catalyst, which can result in higher catalytic activity during the dehydrogenation reaction (e.g., at the beginning of the reaction). For example, without being bound by any particular theory, it may be that pretreating a catalyst composition comprising ZrO₂ can increase the Zr surface-active sites as compared to a catalyst composition that has not been pretreated. In embodiments, a pretreated catalyst composition containing ZrO₂ comprises more surface-active sites (e.g., Zr_{cus}) than a catalyst composition containing ZrO₂ that has not been pretreated.

In embodiments, a pretreated catalyst composition can be formed by contacting the catalyst composition with a pretreatment gas under certain conditions. The pretreatment gas can include a reducing agent comprising at least one of H₂, carbon monoxide (CO), ammonia and methane (CH₄). In embodiments, the pretreatment gas can further include an inert gas comprising at least one of nitrogen (N₂), helium (He) and Argon (Ar). In embodiments, the pretreatment gas can comprise the reducing agent at a concentration of about 1 mol % to about 10 mol %, or about 2 mol %, or about 4 mol %, or about 6 mol %, or about 8 mol %, or about 10 mol %. In certain embodiments, the pretreatment gas is H₂ and can include about 1 mol % to about 10 mol % H₂, or about 2 mol % H₂, or about 4 mol % H₂, or about 6 mol % H₂, or about 8 mol % H₂, or about 10 mol % H₂.

During the pretreatment, the pretreatment gas and/or the catalyst composition can be at a temperature of at least about 850 K, or at least about 860 K, or at least about 870 K, or at least about 873 K, or at least about 880 K, or at about 870 K, or at about 871 K, or at about 872 K, or at about 873 K, or at about 874 K, or at about 875 K. The pretreatment gas can be in contact with the catalyst composition for about 1 h to about 24 h, or about 2 h to about 22 h, or about 3 h to about 20 h, or about 5 h to about 15 h, or about 8 h to about 12 h, or about 1 h, or about 2 h, or about 3 h, or about 4, hour or about 5 h.

According to various embodiments, catalyst compositions as disclosed herein can be in the form of a plurality of units. The plurality of units can include, but are not limited to, particles, powder, extrudates, tablets, pellets, agglomerates, granules and combinations thereof. The plurality of units can have any suitable shape known to those of ordinary skill in the art. Non-limiting examples of shapes include round, spherical, spheres, ellipsoidal, cylinders, hollow cylinders, four-hole cylinders, wagon wheels, regular granules, irregular granules, multilobes, trilobes, quadrilobes, rings, monoliths and combinations thereof.

The plurality of units can be formed by any suitable method known to those of ordinary skill in the art. Non-limiting examples of methods for shaping and forming a plurality of units (e.g., from a mixture of catalyst materials) include, extrusion, spray drying, pelletization, agglomeration, oil drop, and combinations thereof. In one embodiment, the plurality of units can be formed by pressing a powder into wafers (e.g., at about 690 bar, for about 0.05 h), crushing the wafers and then sieving the resulting aggregates to retain a mean aggregate size of about 100 μm to about 250 μm, or about 1.5 mm to about 5 mm, or about 80 mesh to about 140 mesh.

In certain embodiments, the plurality of units can have a size of less than about 1,000 μm, or less than about 750 μm, or less than about 500 μm, or less than about 300 μm, or less than about 250 μm, or less than about 225 μm, or less than about 200 μm, or less than about 190 μm, or less than about 180 μm, or less than about 150 μm, or less than about 100 μm, or less than about 10 μm as measured by any suitable method known to those of ordinary skill in the art. In embodiments, the plurality of units can have a size of about 170 μm to about 250 μm, or about 80 mesh to about 140 mesh. In further embodiments, the plurality of units have a mean size of about 1.5 mm to about 15.0 mm, or about 1.5 mm to about 12 mm, or about 1.5 mm to about 10 mm, or about 1.5 mm to about 8.0 mm, or about 1.5 mm to about 5.0 mm. Particle size can be measured using any suitable method known to those of ordinary skill in the art. For example, particle size can be measured using ASTM D4438-85 (2007) and ASTM D4464-10, both of which are incorporated herein by reference in their entirety.

According to embodiments, catalyst compositions as described herein may be comprised in a kit. The kit can include the catalyst composition as described above and instructions for pretreating the catalyst composition. The instructions can comprise the following elements: 1) place the catalyst composition in a chamber and/or reactor; 2) heat the pretreatment gas, the chamber, the reactor and/or the catalyst composition to a temperature of at least about 850 K, or at least about 860 K, or at least about 870 K, or at least about 873 K, or at least about 880 K, or at about 870 K, or at about 871 K, or at about 872 K, or at about 873 K, or at about 874 K, or at about 875 K; 3) introduce the pretreatment gas into the chamber and/or reactor and contact the catalyst composition with the pretreatment gas for about 0.1 h to about 24 h, or about 0.5 h to about 22 h, or about 1.0 h to about 20 h, or about 2.5 h to about 15 h, or about 5 h to about 12 h, or about 0.1 h, or about 0.2 h, or about 0.5 h, about 1 h, or about 2 h, or about 3 h, or about 4, hour or about 5 h.

According to embodiments, the kit may include the catalyst composition together with instructions for using the catalyst composition in a light alkane (or light alkene) dehydrogenation process. The catalyst composition can be pretreated or may not be pretreated in accordance with embodiments herein. If not pretreated, the kit can further include instructions for pretreating the catalyst composition as described above. The instructions for using the catalyst composition can comprise the following elements: 1) place the catalyst composition in a reactor; 2) introduce the light alkane gas (and/or light alkene gas) together with H₂ into the reactor; and 3) contact the light alkane gas (and/or light alkene gas) and the H₂ with the catalyst composition. Optionally, the instructions may further include 4) recover the dehydrogenated (i.e., alkene or alkadiene) gas.

The kits discussed above can include suitable details and instructions for using the catalyst composition safely and productively. Non-limiting examples of such details and instructions can include how to load the catalyst composition into the reactor, how to pre-treat the catalyst composition, if necessary, before starting the reaction, the starting temperatures and gas composition for bringing the catalyst composition on-stream, the regeneration procedures, how to unload the catalyst composition from the reactor and combinations thereof.

### Methods of Preparing the Catalyst Compositions

According to various embodiments, disclosed herein are methods of preparing catalyst compositions as described above. A catalyst composition comprising ZrO₂ can be prepared using a hydrothermal approach. In embodiments, the catalyst composition comprising ZrO₂ can be synthesized by dissolving an amount of ZrO(NO₃)₂xH₂O in an amount of water (e.g., deionized water) at a weight ratio of water to ZrO(NO₃)₂xH₂O of about 1:1 to about 5:1, or about 2:1 to about 4:1, or about 2:1, or about 2.3:1, or about 2.4:1, or about 3:1, or about 4:1. Separately urea can be dissolved in water at a weight ratio of water to urea of about 1:2 to about 4:1, or about 1:1 to about 3:1, or about 1.5:1 to about 2:1, or about 1.1:1, or about 1.2:1, or about 1.3:1, or about 1.4:1. The ZrO(NO₃)₂xH₂O and urea solutions can be mixed together at a temperature of about 20° C. to about 25° C. and then transferred to an autoclave formed of a suitable material (e.g., stainless steel or other metallurgy, Teflon® or other suitable polymer). The mixture can be crystallized in the autoclave at an elevated temperature, for example, a temperature of about 300 K to about 500 K, or about 320 K to about 480 K, or about 350 K to about 460 K, or about 400 K, or about 450 K, or about 451 K, or about 452 K, or about 453 K, or about 454 K, or about 455 K under autogenous pressure for a period of time, for example, about 2 h to about 36 h, or about 5 h to about 32 h, or about 10 h to about 24 h, or about 12 h to about 20 h, or about 18 h, or about 19 h, or about 20 h, or about 21 h, or about 22 h. The crystalline precipitate can be washed with deionized water, for example, at a ratio of about 50 mL to about 250 mL of deionized water per gram of catalyst, or about 100 mL deionized water per g catalyst and subsequently dried under air at a temperature of about 300 K to about 500 K, or about 325 K to about 450 K, or about 350 K to about 400 K, or about 380 K, or about 381 K, or about 382 K, or about 383 K, or about 384 K, or about 385 K, for a period of about 6 h to about 24 h, or about 8 h to about 22 h, or about 10 h to about 20 h, or about 12 h to about 18 h. In embodiments, the crystalline precipitate can be calcined according to any suitable method known to those of ordinary skill in the art. For example, the crystalline precipitate can be calcined in flowing dry air (e.g., zero grade) at a flow rate of about 0.5 cm³ s⁻¹ to about 2.00 cm³ s⁻¹, or about 1.67 cm³ s⁻¹ and a temperature of about 700 K to about 1,000 K, or about 750 K to about 950 K, or about 800 K to about 900 K, or about 870 K, or about 871 K, or about 872 K, or about 873 K, or about 874 K, or about 875 K, or about 923 K, for about 1 h to about 6 h, or about 3 h.

In embodiments, a catalyst composition comprising yttrium stabilized zirconium oxide can be prepared. The catalyst composition can be synthesized by combining ZrO(NO₃)₂ with Y(NO₃)₃, NH₄OH and H₂O to form a mixture. The mixture can be transferred to an autoclave to be crystallized at an elevated temperature of about 250 K to about 500 K, or about 275 K to about 480 K, or about 290 K to about 460 K, or about 295 K, or about 296 K, or about 297 K, or about 298 K, or about 299 K, or about 300 K, or about 301 K under autogenous pressure for a period of time, for example, about 2 h to about 36 h, or about 5 h to about 32 h, or about 10 h to about 24 h, or about 12 h to about 20 h, or about 10 h, or about 11 h, or about 12 h, or about 13 h, or about 14 h. The crystalline precipitate can be washed with deionized water, for example, at a ratio of about 50 mL to about 250 mL of deionized water per gram of catalyst, or about 100 mL deionized water per g catalyst, and subsequently dried under air at a temperature of about 300 K to about 500 K, or about 325 K to about 450 K, or about 350 K to about 400 K, or about 380 K, or about 381 K, or about 382 K, or about 383 K, or about 384 K, or about 385 K, for a period of about 6 h to about 24 h, or about 8 h to about 22 h, or about 10 h to about 20 h, or about 12 h to about 18 h. In embodiments, the crystalline precipitate can be calcined according to any suitable method known to those of ordinary skill in the art. For example, the crystalline precipitate can be calcined in flowing dry air (e.g., zero grade) at a flow rate of about 0.5 cm³ s⁻¹ to about 2.00 cm³ s⁻¹, or about 1.67 cm³ s⁻¹ and a temperature of about 700 K to about 1,000 K, or about 750 K to about 950 K, or about 800 K to about 900 K, or about 870 K, or about 871 K, or about 872 K, or about 873 K, or about 874 K, or about 875 K, or about 923 K, for about 1 h to about 6 h, or about 3 h.

According to embodiments, methods of preparing the catalyst composition can further include pretreating the catalyst composition in a pretreatment gas as discussed above. For example, the catalyst composition can be subjected to a reductive pretreatment, for example, with a pretreatment gas comprising at least one of H₂, carbon monoxide (CO), light alkanes, propane (C₃H₈), alkenes, propene (C₃H₆) and H₂ species present as reactant and products of the dehydrogenation reaction. In embodiments, the catalyst composition can be pretreated with H₂ at a temperature of about 800 K to about 1,000 K, or about 850 K to about 900 K, or about 873 K. The pretreated catalyst composition can include more surface-active sites (e.g., Zr_{cus} surface-active sites) than a catalyst composition that has not been pretreated.

### Methods of Using the Catalyst Compositions

Further described are methods of using catalyst compositions according to embodiments. In embodiments, the catalyst compositions can be used in the dehydrogenation of light alkane gas to form alkenes. In embodiments, the methods can also be used in the dehydrogenation of light alkene gas to form alkadienes. The ZrO₂ present in the catalyst compositions is the active catalyst material in the dehydrogenation reactions as disclosed herein. In contrast to, e.g., the Oleflex® process where ZrO₂ is used as an inactive support for the active Pt—Sn catalyst materials, the ZrO₂ in the present catalyst compositions is the active catalyst material. It is believed that ZrO₂ has not before been used as the active catalyst material for the dehydrogenation of light alkane (and/or light alkene) gas according to embodiments herein.

In embodiments, when preparing to dehydrogenate a light alkane gas, the catalyst composition (e.g., at a weight hourly space velocity of about 5.5 h⁻¹ to about 0.05 h⁻¹, or about 5.4 h⁻¹ to about 0.054 h⁻¹, or about 2.7 h⁻¹ to about 1.8 h⁻¹, or about 5.0 h⁻¹ to about 0.1 h⁻¹) can be placed within a reactor and held at an about constant temperature using a furnace and a temperature controller (e.g., a Watlow Series 96). The reactor can be any suitable reactor known to those of ordinary skill in the art. Non-limiting examples include a U-shape quartz reactor (e.g., with an inner diameter of about 11.0 mm), a packed tubular reactor, a catofin-type reactor, a fluidized bed reactor, a fixed bed reactor and a moving bed reactor. The furnace may be any suitable furnace known to those of ordinary skill in the art. Non-limiting examples include a single zone furnace (e.g., by National Element Inc., Model No. BA-120), a batchwise furnace or a quartz tube furnace.

Prior to dehydrogenation, the catalyst composition can be treated in a flowing oxygen gas (O₂) and helium (He) mixture at a molar ratio of O₂ of 20:1 to about 30:1, or about 22:1 to about 28:1, or about 24:1 and heating the flowing gas mixture to a temperature of about 800 K to about 1,000 K, or about 850 K to about 900 K, or about 873 K at a rate of about 0.1 K s⁻¹ to about 1 K s⁻¹, or about 0.167 K s⁻¹. Treating the catalyst composition with the flowing O₂ and He gas mixture can be for a period of about 0.5 h to about 8 h, or about 1 h to about 4 h, or about 1 h, or about 2 h. Subsequently, the reactor can be purged with flowing inert gas (as defined above), steam, or by vacuum (e.g., 2 cm³ g⁻¹ s⁻¹, ultra-high purity) to remove residual O₂ within the reactor.

The light alkane gas (and/or light alkene gas) can be introduced to the reactor in the presence of the catalyst composition. According to embodiments, the light alkane gas (and/or light alkene gas) can comprise any one of a C₂ to C₅ straight or branched alkane and mixtures thereof. In embodiments, the light alkane gas (and/or light alkene gas) can comprise at least one of ethane, propane, n-butane, isobutane, pentane and mixtures thereof. In embodiments, a portion of the effluent from the reactor can be recycled to the gas inlet and combined with fresh feed gas. The effluent can comprise alkenes, for example, at least one of ethene, pentene, butene, isobutene and pentene, and unreacted light alkanes comprising at least one of ethane, propane, n-butane, isobutane and pentane. In embodiments, the light alkane gas (and/or light alkene gas) can be mixed with an inert gas (e.g., steam, He, N₂, Ar) at a molar ratio of about 1:2 to about 2:1, or about 1:1. In embodiments, a vacuum pump can be used to lower the pressure of the reactants while maintaining the total pressure above, for example, 1 bar to allow convective flow when the exit pressure is atmospheric.

Methods of dehydrogenating light alkane gas (and/or light alkene gas) can include co-feeding H₂ with the light alkane gas (and/or light alkene gas) in the presence of the catalyst composition. The catalyst composition can comprise ZrO₂ according to various embodiments described herein. Adding or co-feeding the H₂ with the light alkane gas (and/or light alkene gas) can include introducing the H₂ at a pressure of about 1 kPa to about 100 kPa, or about 5 kPa to about 75 kPa, or about 10 kPa to about 50 kPa, or about 30 kPa to about 50 kPa while dehydrogenating the light alkane gas. In embodiments, the H₂ can be added to the light alkane gas (and/or light alkene gas) at a molar ratio of H₂ to light alkane gas (and/or light alkene gas) of about 1:100 to about 1:1. In embodiments, the H₂ and/or reactor can be at a temperature of about 500 K to about 1000 K, or about 550 K to about 950 K, or about 600 K to about 900 K, or about 700 K to about 900 K.

According to embodiments, the method of dehydrogenating a light alkane gas (and/or light alkene gas) in the presence of H₂ and a catalyst composition as described herein can provide a dehydrogenation rate per mass of the catalyst composition of about 0.5 mol kg⁻¹ h⁻¹ to about 10.0 mol kg⁻¹ h⁻¹, or about 0.6 mol kg⁻¹ h⁻¹ to about 8.3 mol kg⁻¹ h⁻¹. The light alkane gas (and/or light alkene gas) dehydrogenation rate and cracking rate can be determined by analyzing the effluent stream from the reactor using gas chromatography (e.g., by an Agilent® 1540A gas chromatograph) with flame ionization detection (FID) (e.g., a GC fitted with a GS-GASPRO column) after chromatographic separation. In embodiments, the light alkane gas (and/or light alkene gas) dehydrogenation rate and the cracking rate can be normalized by the mass of the catalyst composition (e.g., in mol kg⁻¹ h⁻¹). In embodiments, the light alkane gas (and/or light alkene gas) dehydrogenation rate and cracking rate can be determined at a temperature of 873 K and 823 K. The temperature can be measured using any suitable method known to those of ordinary skill in the art. In embodiments, the temperature can be measured with a thermocouple (e.g., a K-type thermocouple by Omega®) and the reactor temperature can be determined from a thermocouple placed in contact with an outer tube surface (e.g., made of metal, quartz, etc.) at the catalyst bed midpoint.

In embodiments, when used in a dehydrogenation reaction as described above, catalyst compositions as disclosed herein can provide improved stability over other known catalyst compositions for the dehydrogenation of light alkanes (and/or light alkenes). The half-life of the catalyst composition in the dehydrogenation reaction can be measured using any suitable method known to those of ordinary skill in the art. In embodiments, the term “half-life of the catalyst composition” can refer to the number of days or hours after which the catalyst device has a dehydrogenation rate (DR) that is 50% lower than an initial or maximum dehydrogenation rate (DRᵢ) value produced by the catalyst composition at the start (or soon after the start upon stabilization) of the catalyst composition's operation (e.g., the half-life of the catalyst composition can be based on the dehydrogenation reaction rate as a function of time). In embodiments, the half-life of the catalyst composition is related to the weight hourly space velocity (WHSV), which is the hourly mass feed flow rate per catalyst mass (h⁻¹) in the reactor. In embodiments, the catalyst composition can have a half-life of about 1 h to about 50 h, or about 6 h to about 46 h when the WHSV is about 5.4 h⁻¹ to about 0.054 h⁻¹.

The half-life of the catalyst composition can also be evaluated when aging the composition under different conditions. The DR can be calculated based on Formula I:

$$DR = 60 \cdot ( {k}_{e} - {k}_{n} ) \cdot V \qquad Formula I$$

wherein kₑ represents the total decay constant, kₙ represents natural decay constant, V represents the chamber volume in m³, and (kₑ−kₙ) is calculated based on Formula II:

$$( {k}_{e} - {k}_{n} ) \cdot t = - ln ( {C}_{t} / {C}_{0} ) \qquad Formula II$$

wherein t represents the total testing time, Cₜ represents the concentration at time t in mg/m³, and C₀ represents the concentration at time t=0 in mg/m³.

To determine the half-life of the catalyst composition, testing can begin by obtaining the DR value produced by the catalyst composition at the start of the catalyst composition's operation or soon after the start of the catalyst composition's operation once the DR has stabilized (t=0), also known as DR₀. Subsequently, the catalyst composition can be optionally subjected to an Acceleration Test. An “Acceleration Test” refers to an extreme condition that may impact or deteriorate the efficacy of the catalyst composition more rapidly, such as no H₂ gas co-feed or pre-treatment, co-feeding with O₂, introducing a pollutant or continuous generation of pollutants. The optional Acceleration Test results can allow the estimation of the catalyst composition's life span under real-life conditions. Following the optional Acceleration Test, the catalyst composition is then aged under real-life conditions.

After the catalyst composition is aged for eight hours under real-life conditions, another sample is taken to obtain the DR value at t=n, also known as DRₙ. If the DRₙ value is greater than 50 percent of the DR₀ value, then the catalyst composition is considered as still operable and the testing continues by repeating the optional acceleration test, aging the catalyst device under real-life conditions, and measuring the DRₙ value after each subsequent cycle. Once the DRₙ value is lower or equal to 50 percent of the DR₀ value, the life span of the catalyst device is deemed to have ended and the overall alkene mass (AM) generated by the catalyst composition is calculated.

The above-described methods of using the catalyst compositions to dehydrogenate light alkane gas, can also be used to dehydrogenate a light alkene gas to form alkadienes. A light alkene gas can include C₂-C₅ branched or straight alkenes. In embodiments, two reactors can be configured in series, the first for dehydrogenating light alkane gas and the second for dehydrogenating the light alkene gas.

### EXAMPLES

### Example 1—Crystal Structure of ZrO₂

A ZrO₂ catalyst composition was prepared using a hydrothermal approach. To synthesize the ZrO₂, 12.7 g ZrO(NO₃)₂xH₂O (99%, Aldrich) were dissolved in 30 mL deionized H₂O and 21.6 g urea were dissolved in 30 mL deionized H₂O separately. The resulting solutions were mixed together at room temperature and then transferred into a Teflon lined stainless-steel autoclave. Crystallization was performed at 453 K under autogenous pressure for 20 h. After completion of the crystallization process, the precipitate was washed thoroughly with 500 ml deionized water and then dried under air at 383 K overnight.

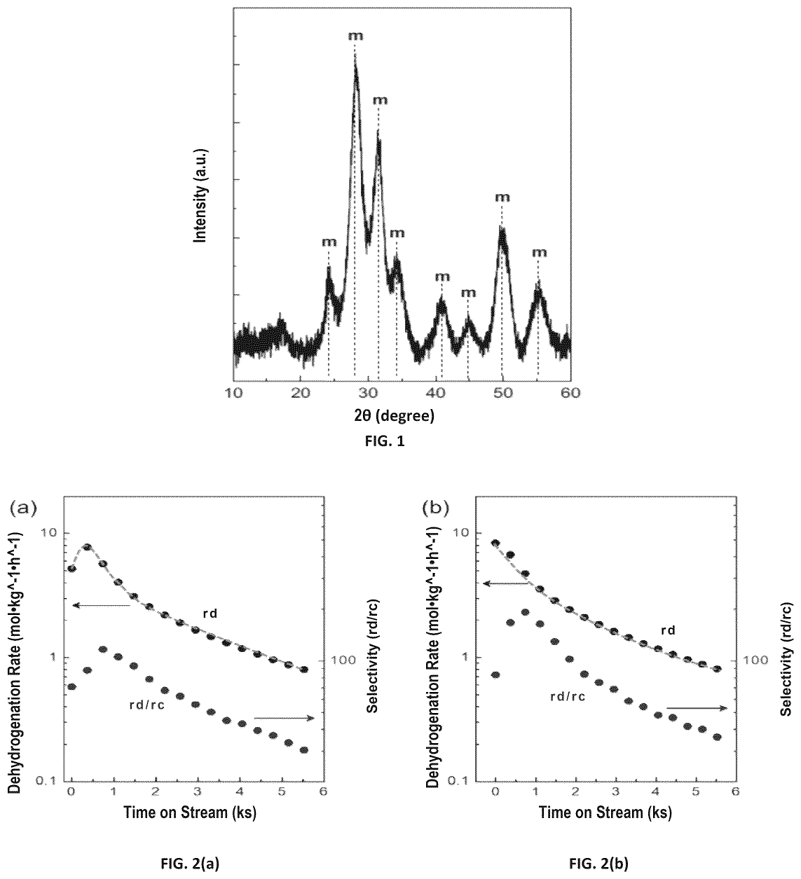

The crystal structure of the resulting ZrO₂ catalyst composition was measured using X-ray diffraction. The XRD pattern of ZrO₂ catalyst composition is shown in FIG. 1. The data herein indicates that catalyst compositions as described can retain the tetragonal phase at all temperatures of treatment while providing higher dehydrogenation rates.

### Example 2—Effect of Pretreatment on Initial C₃H₈ Dehydrogenation Rate (r_{d}) at 873 K

The effect of pretreating the ZrO₂ catalyst composition as prepared in Example 1 on initial propane dehydrogenation (PDH) rate (r_{d}) at 873 K was evaluated. In order to compare the effect of oxidative and reductive pretreatments on PDH activity of the ZrO₂ catalyst composition, the catalyst was pretreated in a 4 mol % O₂/He mixture at 873 K for 2 h and separately in a 10 mol % H₂/He mixture at 873 K for 2 h prior to the PDH reaction.

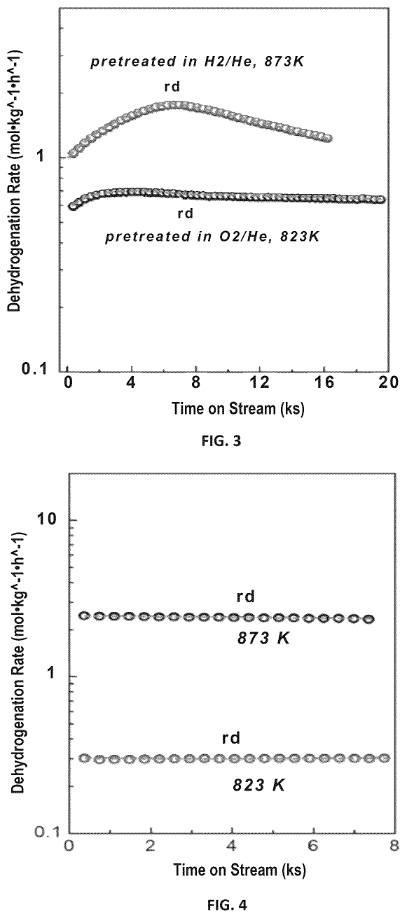

The O₂/He pretreated ZrO₂ catalyst was introduced into the reactor. During the propane dehydrogenation reaction, the propane feed gas was at a pressure of 13.7 kPa and a temperature of 873 K and the catalyst composition was at a temperature of 873 K. As shown in FIG. 2A, an initial increase in dehydrogenation rate (per mass) was observed within 6 min of the reaction. The dehydrogenation rate increased from 5.2 mol kg-1 h⁻¹ to 7.8 mol kg⁻¹ h⁻¹ while the selectivity of the PDH reaction (ratio between dehydrogenation and cracking rate (r_{d}/re)) also increased from 71 to 88 during this 6 min activation period. A monotonic decrease in dehydrogenation rate and selectivity was observed after the initial activation period indicating the deactivation of ZrO₂ catalyst. Without being bound by any particular theory, the increase in dehydrogenation rate during the initial activation period may indicate the presence of an activation effect. Because the surface lattice oxygen was removed, Zr surface active sites possibly were generated leading to a higher dehydrogenation rate.

After the initial activation period of 6 min, the dehydrogenation rate and selectivity (r_{d}/r_{c}) of ZrO₂ catalyst decreased with time. The deactivation process follows a first-order deactivation mechanism as shown in equation 1:

$$ln ( \frac{{r}_{i}}{{r}_{0}} ) = - {k}_{d} t \qquad ( 1 )$$

where r₀ is the maximum dehydrogenation rate at the beginning of PDH reaction or during the initial activation period, rᵢ is the dehydrogenation rate at different time on stream and ka is the deactivation constant. The half-life of the catalyst is defined in equation 2, which is time elapsed as the catalyst deactivates to one-half of its initial dehydrogenation rate:

$${t}_{0.5} = \vert \frac{ln ( 0.5 )}{{k}_{d}} \vert \qquad ( 2 )$$

FIG. 2A shows that after the initial activation period during PDH reaction at 873 K, a half-life of 0.28 h can be observed for the ZrO₂ catalyst pretreated in O₂/He at 873 K and the deactivation is mainly caused by the formation of carbonaceous species on the surface, as the spent catalyst exhibited black color. This observation confirms previous observations (see, e.g., Zhang, et al., Control of Coordinatively Unsaturated Zr Sites in ZrO₂ for Efficient C—H Bond Activation, Nature Communications, 9 (1), 3794 (2018)) that ZrO₂ catalysts during PDH reaction suffer from rapid deactivation with a catalyst's half-life of 0.2 h during PDH reaction at 873 K under 40 kPa C₃H₈ and require regeneration on a regular basis.

A fresh catalyst was treated in flowing 10% H₂/He mixture at 873 K for 2 h. As shown in FIG. 2B, for the ZrO₂ catalyst pretreated in the H₂/He, the initial activation period was not observed during the PDH reaction at 873 K. The initial dehydrogenation rate was 8.3 mol kg⁻¹ h 1, which is comparable to the highest dehydrogenation (i.e., 7.8 mol kg⁻¹ h⁻¹) during initial activation period on ZrO₂ treated in 4% O₂/He at 873 K. This indicates that a reductive pretreatment by H₂ can activate ZrO₂ to a similar extent as an oxidative pretreatment during the initial activation period. The dehydrogenation rate on ZrO₂ treated in H₂/He at 873 K decreased monotonically with time on stream with a half-life of 0.32 h, a value similar to that on ZrO₂ treated in O₂/He at 873 K (0.28 h). This indicates that the deactivation behavior is not affected by pretreatment methods (oxidative or reductive). Based on the above observation, it was determined that H₂ can activate the ZrO₂ catalyst through H₂/He pretreatment before the PDH reaction.

### Example 3—Dehydrogenation Rate Per Mass of Catalyst (r_{d}) during a C₃H₈ dehydrogenation reaction on a pretreated ZrO₂ Catalyst

The dehydrogenation rate per mass of catalyst (r_{d}) during a PDH reaction on a pretreated ZrO₂ catalyst was determined. A ZrO₂ catalyst composition as prepared in Example 1 was pretreated with O₂ at a pressure of 4 kPa and a temperature of 823 K for 2 h. Separately, another ZrO₂ catalyst composition as prepared in Example 1 was pretreated in H₂ at a pressure of 10 kPa and a temperature of 873 K for 2 h. The effect of pretreatment was then studied for a PDH reaction at 823 K on the ZrO₂ catalyst. During the PDH reaction, propane was introduced to the reactor at a pressure of 13.7 kPa and a temperature of 823 K.

As shown in FIG. 3, after the catalyst was treated in O₂/He at 823 K, an initial activation effect was observed, which lasted for 70 min with an increase in dehydrogenation rate from 0.6 mol kg-1 h⁻¹ to 0.7 mol kg⁻¹ h⁻¹. After the initial activation period, the catalyst started to deactivate at a much slower rate as compared to the deactivation process during the PDH reaction at 873 K (Example 2). Moreover, for the PDH reaction at 823 K, there is no contribution to cracking rate from the ZrO₂ catalyst and all cracking products detected can be attributed to homogenous gas phase reactions of propane, which can occur even in empty reactors at these temperatures. The half-life for the catalyst pretreated with O₂/He during PDH reaction at 823 K was 34 h, which is 120 times higher than the half-life during the PDH reaction at 873 K (0.28 h). The ZrO₂ catalyst treated in H₂/He at 873 K had a higher initial dehydrogenation rate (1.1 mol kg⁻¹ h⁻¹) compared with the catalyst (0.7 mol kg⁻¹ h⁻¹) treated in O₂/He at 823 K during PDH reaction at 823 K. These data demonstrate the activation effect of the H₂ pretreatment (FIG. 3).

Different from the PDH reaction at 873 K on the ZrO₂ catalyst treated in H₂/He at 873 K where no initial activation period can be observed, the PDH reaction at 823 K on the ZrO₂ catalyst treated in H₂/He at 873 K exhibited an initial activation period (110 min, up to 1.8 mol kg⁻¹ h⁻¹). This indicates that the pre-reduced ZrO₂ can be further activated during the PDH reaction by propane-derived species. A 6 h half-life can be observed for ZrO₂ treated in H₂/He at 873 K during PDH reaction at 823 K, as compared to the ZrO₂ catalyst treated in O₂/He at 823 K (34 h).

### Example 4—Effect of Co-Feeding H₂ on the stability of ZrO₂ Catalyst at 873 K and 823 K

The effect of co-feeding H₂ with the light alkane gas (e.g., propane) during a dehydrogenation reaction in the presence of a ZrO₂ catalyst (as prepared in Example 1) was evaluated. The dehydrogenation rate per mass (r_{d}) of ZrO₂ catalyst pretreated with O₂/He, with additional H₂ present during the PDH reaction at different temperatures, was determined. During the PDH reaction, H₂ was introduced to the reactor at a pressure of 12.3 kPa and C₃H₈ was co-introduced at a pressure of 13.7 kPa. The reaction was performed at a temperature of 873 K and separately at a temperature 823 K.

Prior to the PDH reaction, the catalyst was pretreated in O₂/He at corresponding temperatures corresponding the PDH reaction temperatures (i.e., 823 K, 873 K). As the H₂ was co-fed into the reaction gas stream comprising C₃H₈, an inhibition effect on dehydrogenation rate was observed at 823 K. As shown in FIG. 4, the initial dehydrogenation rate at 823 K decreased from 0.7 mol kg⁻¹ h⁻¹ to 0.31 mol kg⁻¹ h⁻¹. Also shown in FIG. 4, the initial dehydrogenation rate at 873 K decreased from 5.2 mol kg⁻¹ h⁻¹ to 2.5 mol kg⁻¹ h⁻¹.

It was also found that there was no contribution to the cracking rate from the ZrO₂ catalyst during the PDH reaction with the additional H₂ at a pressure of 12.3 kPa at temperatures of 823 K and 873 K. The lack of contribution to the cracking rate at 873 K indicates that co-feeding with H₂ can improve the selectivity of the ZrO₂ catalyst towards C₃H₆.

The stability of the ZrO₂ catalyst improved in the PDH reaction when additional H₂ was co-fed with the propane at a pressure of 12.3 kPa and a temperature of 873 K. Indeed, the catalyst half-life was 46 h as compared to 0.28 h during the PDH reaction at 873 K without co-feeding with H₂. At 823 K, there was no observable deactivation during the PDH reaction under the presence of additional H₂ at a pressure of 12.3 kPa. Because H₂ pretreatment can activate the catalyst as demonstrated in Example 3, it is possible that by co-feeding H₂ during the PDH reaction, Zr_{cus} surface-active sites may be generated or maintained continuously so that any formation of carbonaceous species does not affect the catalytic activity.

FIG. 5 shows the CO₂ generation rate during a re-oxidation process after a PDH reaction with and without the presence of additional H₂ at 873 K on the ZrO₂ catalyst. During the re-oxidation, a mixture of 4% O₂/He was provided at a rate of 2 cm³ g⁻¹ s⁻¹ and a temperature of 873 K. By comparing the total amount of CO₂ generated during the re-oxidation process, it was found that by co-feeding the H₂ with the propane feed gas, the formation of carbonaceous species could be inhibited.

After the PDH reaction at 873 K on the ZrO₂ catalyst pretreated with O₂/He, the total amount of CO₂ generated during the subsequent re-oxidation process was 2.6 mmol g_{cat}⁻¹, which corresponds to a surface carbon density of 35 C-atoms/nm². Considering the surface Zr—O pair density of 7 nm⁻², the formation of carbonaceous species is likely to cover the active sites on ZrO₂ catalyst surface, which can eventually lead to deactivation. After the PDH reaction with additional 12.3 kPa H₂ at 873 K on ZrO₂ catalyst with O₂/He pretreatment, only 0.5 mmol g_{cat}⁻¹ CO₂ was generated during subsequent re-oxidation process, which corresponds to a surface carbon density of 6.6 nm⁻² (FIG. 5). The presence of additional H₂ at a pressure of 12.3 kPa during the PDH reaction at 873 K, led to a greater than five-fold decrease in the surface density of carbonaceous species as compared to the PDH reaction at 873 K without additional H₂. Together with the observed activation effect on the ZrO₂ catalyst during the PDH reaction after H₂ pretreatment, the stabilization of ZrO₂ during the PDH reaction by co-feeding H₂ may be attributable to continuous generation of surface-active sites while the formation of carbonaceous species is effectively inhibited. Without being bound by any particular theory, these data seem to indicate that the role of H₂ is to continuously remove the C as it forms at a rate higher than that achieved by the H₂ formed in situ by the dehydrogenation reaction. These two effects of pre-treatment and co-feeding with H₂ lead to a stable ZrO₂ catalyst during the PDH reaction with high selectivity towards propene.

### Example 5—Dependence of Dehydrogenation Rate on H₂ and C₃H₈

The dependence of the dehydrogenation rate on H₂ and C₃H₈ was evaluated. The dehydrogenation rate per mass of catalyst (r_{d}) was determined during a PDH reaction with additional H₂ at a pressure of 12.3 kPa and a temperature of 873 K on the ZrO₂ catalyst as a function of C₃H₈ partial pressure. Due to the stabilization effect on the activity of the ZrO₂ catalyst during the PDH reaction with additional H₂, the dependence of the dehydrogenation rate on the partial pressure of C₃H₈ could be accurately determined at 873 K under the presence of additional H₂ at a pressure of 12.3 kPa for a ZrO₂ catalyst pretreated in O₂/He at 873 K. As shown in FIG. 6, the dehydrogenation rate increased proportionally with increasing C₃H₈ partial pressure while there was no contribution to cracking rate from the ZrO₂. This trend indicates that at 873 K the PDH reaction occurs on the surface with low coverages of C₃H₈-derived species, which are either weakly-bound or scavenged in fast subsequent reactions after they formed.

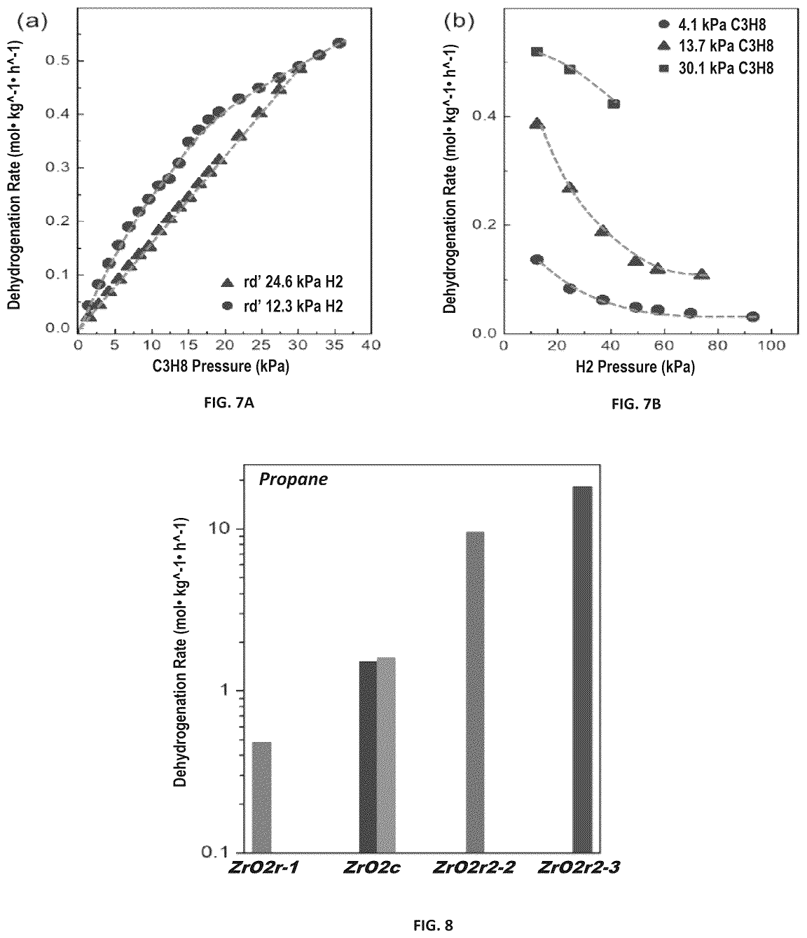

FIG. 7A shows the dehydrogenation rate per mass of catalyst (r_{d}) during a PDH reaction where the propane gas was co-fed with H₂ at a pressure of 12.3 kPa or 24.6 kPa and a temperature of 823 K on the ZrO₂ catalyst as a function of C₃H₈ partial pressure. FIG. 7B shows the dehydrogenation rate per mass of catalyst (r_{d}) during the PDH reaction where the propane gas was introduced at a pressure of 4.1 kPa, 13.7 kPa and 30.1 kPa and a temperature of 823 K on the ZrO₂ catalyst as a function of H₂ partial pressure.

The dependence of dehydrogenation rate on C₃H₈ and H₂ partial pressure at 823 K was determined for a ZrO₂ catalyst pretreated in O₂/He at 823 K. Under 12.3 kPa of additional H₂, an inhibition of dehydrogenation rate at elevating C₃H₈ partial pressure was observed at 823 K. This indicates that under additional H₂ at a pressure of 12.3 kPa, the catalyst surface has become saturated with C₃H₈-derived intermediates at high C₃H₈ partial pressure (FIG. 7A), which can inhibit the dehydrogenation rate.

As the partial pressure of additional H₂ increased to 24.6 kPa, the dehydrogenation rate increased proportionally with increasing C₃H₈ partial pressure at 823 K, indicating that under additional H₂ at a pressure of 24.6 kPa, the C₃H₈-derived intermediates can undergo fast subsequent reaction on the catalyst surface and the dehydrogenation rate will not be inhibited. No contribution to cracking rate from the ZrO₂ was observed during the above mentioned PDH reaction under 12.3 kPa and 24.6 kPa of additional H₂.

As shown in FIG. 7B, the dependence of dehydrogenation rate on H₂ partial pressure was studied under 4.1 kPa, 13.7 kPa and 30.1 kPa C₃H₈ at 823 K. An inhibition effect on the dehydrogenation rate was observed for all three C₃H₈ partial pressures at increasing H₂ partial pressure and the dehydrogenation rate eventually became constant with an H₂ partial pressure higher than 58 kPa. There are two possible origins for the H₂ inhibition effect: 1) either the additional H₂ is reversing the C—H bond activation step by reacting with the formed alkyl group to re-form C₃H₈; or 2) it is increasing the H-content of the carbonaceous deposit and making it less willing to accept hydrogen from C₃H₈.

### Example 6—Benchmark of PDH Activity on Different ZrO₂ Catalysts

A benchmark of the PDH reaction activity on different ZrO₂ catalysts was determined. The dehydrogenation rate of propane on ZrO₂ catalysts as prepared in Example 1 according to various embodiments was compared with the PDH activity data on ZrO₂ from previous studies (FIG. 8). FIG. 8 shows a comparison of initial dehydrogenation rate of propane among ZrO₂ catalysts in current research: a) ZrO_{2c} dehydrogenation rate after the catalyst has been pretreated in 4 kPa O₂, He balance; b) the dehydrogenation rate after the catalyst been regenerated in 4 kPa O₂, He balance; and c) from references ZrO₂ᵣ₋₁, ZrO₂ᵣ₋₂ and ZrO₂ᵣ₋₃. The PDH reaction was operated with C₃H₈ at a pressure of 40 kPa and a temperature of 823K.

The ZrO₂ catalysts from previous studies were commercial ZrO₂ (by Saint-Gobain) with different crystalline sizes (ZrO₂^{r}-1: 43 nm, ZrO₂^{r}-2: 13 nm, ZrO₂^{r}-3: 9 nm). The dehydrogenation rate at 823 K on the commercial ZrO₂ catalyst increased with decreasing crystalline size, but the selectivity towards propene decreased with decreasing crystalline size. For the ZrO₂^{r}-3 catalyst with a crystalline size of 9 nm, the selectivity towards propene was 98%. For the ZrO₂ catalyst as prepared in Example 1 according to embodiments herein, the dehydrogenation rate at 823 K was higher than that on ZrO₂^{r}-1 and the PDH reaction was highly selective towards propene (no contribution to cracking rate from ZrO₂ was observed after the subtraction of gas phase activity). After the PDH reaction, the ZrO₂ catalyst prepared in accordance with Example 1 and embodiments herein, was treated in 4 kPa O₂ at 823 K for 30 min and the subsequent PDH reaction after regeneration exhibited a dehydrogenation rate comparable with the first PDH activity measurement (FIG. 8), which indicates the ZrO₂ catalyst can be fully regenerated by oxidative treatment. This observation is consistent with previous studies where several PDH reaction-regeneration cycles were performed on ZrO₂ at 823 K and no decrease in initial dehydrogenation rate was observed.

The introduction of additional H₂ during the PDH reaction on ZrO₂ catalyst provided a method to improve the stability of the catalyst through an inhibition of the formation of inactive carbonaceous species. This inhibition can lead to higher stability of the catalyst. No contribution to cracking rate from the ZrO₂ catalyst when additional H₂ was present during the PDH reaction at 873 K and all cracking products detected can be fully attributed to homogenous reactions that occur even in an empty reactor. This method may provide a general strategy to improve the stability of oxide-based catalyst during paraffin dehydrogenation reaction.

The preceding description sets forth numerous specific details such as examples of specific systems, components, methods, and so forth, in order to provide a good understanding of several embodiments of the present invention. It will be apparent to one skilled in the art, however, that at least some embodiments of the present invention may be practiced without these specific details. In other instances, well-known components or methods are not described in detail in order to avoid unnecessarily obscuring the present invention. Thus, the specific details set forth are exemplary. Particular embodiments may vary from these exemplary details and still be contemplated to be within the scope of the present invention.

Although the operations of the methods herein are described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent and/or alternating manner.

It is to be understood that the above description is intended to be illustrative, and not restrictive. Many other embodiments will be apparent to those of skill in the art upon reading and understanding the above description. The scope of the invention should, therefore, be determined with reference to the appended claims, along with the full scope of equivalents to which such claims are entitled.

1-36. (canceled)

37. A method for dehydrogenating a light alkane gas, comprising:
combining hydrogen (H₂) with the light alkane gas in the presence of a catalyst composition comprising zirconium oxide (ZrO₂).

38. The method of claim 37, wherein the light alkane gas comprises any one of a C₂ to C₅ straight or branched alkane and mixtures thereof.

39. The method of claim 37, wherein the light alkane gas comprises at least one of ethane, propane, n-butane, isobutane, pentane or mixtures thereof.

40. The method of claim 37, wherein the combining H₂ with the light alkane gas is at a molar ratio of H₂ to light alkane gas of about 1:100 to about 1:1.

41. The method of claim 37, wherein the combining H₂ with the light alkane gas is at a temperature of about 500 K to about 1000 K, or about 700 K to about 900 K.

42. The method of claim 37, wherein the catalyst composition is pretreated with a pretreatment gas.

43. The method of claim 42, wherein the pretreatment gas is at a temperature of at least about 850 K.

44. The method of claim 42, wherein the pretreatment gas comprises a reducing agent.

45. The method of claim 44, wherein the reducing agent comprises at least one of H₂, carbon monoxide (CO), ammonia or methane (CH₄), and optionally, wherein the pretreatment gas further comprises unreacted light alkane gas and at least one alkene.

46. The method of claim 44, wherein the pretreatment gas comprises about 1 mol % to about 10 mol % of the reducing agent.

47. The method of claim 37, wherein the H₂ is present in an amount sufficient to remove surface lattice oxygen from the catalyst composition and generate or reactivate surface-active sites, or wherein the H₂ is present in an amount of H₂ per mass of catalyst of about 1 mol kg⁻¹ h⁻¹ to about 100 mol kg⁻¹ h⁻¹.

48. The method of claim 37, wherein a dehydrogenation rate per mass of the catalyst composition is about 0.5 mol kg⁻¹ h⁻¹ to about 10.0 mol kg⁻¹ h⁻¹.

49. The method of claim 37, wherein the catalyst composition comprises a half-life of about 1 h to about 50 h, or about 6 h to about 46 h.

50. The method of claim 37, wherein the catalyst composition comprises: zirconium oxide (ZrO₂), wherein the catalyst composition is free of at least one of chromium or a precious metal.

51. (canceled)

52. The method of claim 37, wherein the catalyst composition is pretreated by contacting the catalyst composition with a pretreatment gas comprising a reducing agent at a concentration of about 1 mol % to about 10 mol %, and wherein the reducing agent comprises at least one of hydrogen (H₂), carbon monoxide (CO), ammonia or methane (CH₄).

53. The method of claim 37, wherein the catalyst composition is in the form of a plurality of units, wherein the plurality of units comprise at least one of particles, powder, extrudates, tablets, agglomerates, granules, spheres or combinations thereof, and wherein the plurality of units comprise a mean size of about 1.5 mm to about 5.0 mm.

54. The method of claim 37, wherein the catalyst composition comprises about 0.5 wt % to about 50 wt % of a rare earth metal comprising at least one of yttrium (Y), erbium (Er), cerium (Ce), dysprosium (Dy), gadolinium (Gd), lanthanum (La), neodymium (Nd), samarium (Sm), ytterbium (Yb), oxides thereof or mixtures thereof.
